(19) United States
(12) Patent Application Publication
(10) Pub. No.: US 2026/0151881 A1
(43) Pub. Date: Jun. 4, 2026

(54) CONSTANT-VELOCITY, VARIABLE-ANGLE DRIVERS

(71) Applicant: VB SPINE US OPCO LLC, WILMINGTON, DE (US)

(72) Inventors: Joseph Michael Meyer, Terrace Park, OH (US); Larry McClintock, Gore, VA (US)

(21) Appl. No.: 19/407,522

(22) Filed: Dec. 3, 2025

Related U.S. Application Data
(60) Provisional application No. 63/728,008, filed on Dec. 4, 2024.

(51) Int. Cl. B25B 23/00; A61F 2/46

(52) U.S. Cl. CPC B25B 23/0035; A61F 2/4611; A61F 2002/4625

(57) ABSTRACT

A driver for transmitting off-axis torque may include a handle. The driver may include a shaft extending between a first end and a second end. The shaft may be coupled to the handle at the first end of the shaft. The second end of the shaft may define a void. The driver may include an anvil disposed within the void of the second end of the shaft. The driver may include a biasing member disposed within the void of the second end of the shaft between the shaft and the anvil. The biasing member may be configured to bias the anvil against the second end of the shaft. The driver may include a tip engaged with a surface of the anvil. The driver may include a sleeve coupled to the second end of the shaft such that the tip is constrained.

### CROSS-REFERENCE TO RELATED APPLICATIONS

This application claims the benefit of U.S. Provisional Application No. 63/728,008 filed Dec. 4, 2024, the complete disclosure of which is incorporated herein by reference for all purposes.

### TECHNICAL FIELD

Surgical drivers, systems, kits, and methods are provided that are suitable to provide off-axis torque transmission to a surgical tool, implant, or instrument by a surgeon.

### BACKGROUND

Off-axis torque transmission is commonly required in surgical procedures. Conventional solutions include drivers with universal-joints, ball-ended hex and Torx drivers, bevel gear drivers, and drivers with double Cardan, Rzeppa, and Tripod joints. However, each of these solutions do not achieve the desired outcome of off-axis torque transmission at a constant velocity and at a variable angle. For example, universal-joints do not have constant velocity capability because the input and output angular velocities vary depending on the intersection between the input and output shafts. Universal-joint drivers also require through holes, thin walls, and welds, which increases the manufacturing complexity. Also, Ball-ended hex and Torx drivers are typically limited due to shaft misalignment. Further, Rzeppa joint drivers require a cage, which increases the size of the driver and is relatively difficult to manufacture compared to cageless drivers. Accordingly, there has been a long felt need in the field for improved surgical drivers.

### SUMMARY

In some embodiments, a driver for transmitting off-axis torque may include a handle. The driver may include a shaft extending between a first end and a second end. The shaft may be coupled to the handle at the first end of the shaft. The second end of the shaft may define a void. The driver may include an anvil disposed within the void of the second end of the shaft. The driver may include a biasing member disposed within the void of the second end of the shaft between the shaft and the anvil. The biasing member may be configured to bias the anvil against the second end of the shaft. The driver may include a tip engaged with a surface of the anvil. The driver may include a sleeve coupled to the second end of the shaft such that the tip is constrained.

In some embodiments, a driver for transmitting off-axis torque may include a handle configured to transmit torque. The driver may include a shaft extending between a first end and a second end. The shaft may define an aperture between the first end and the second end. The shaft may be coupled to the handle at the first end of the shaft. The second end of the shaft may define a slot. The driver may include an anvil that may define an aperture disposed within the slot of the second end of the shaft. The driver may include a biasing member disposed within the slot of the second end of the shaft. The biasing member may be configured to bias the anvil against the second end of the shaft. The driver may include a tip defining a slot at a first end. The first end may be engaged with a surface of the anvil. The driver may include a rod disposed within the aperture of the shaft. The rod may be configured to fixate the tip at an angle along a longitudinal axis of the shaft. The driver may include a sleeve coupled to the second end of the shaft such that the tip is constrained.

In some embodiments, a method may include coupling a handle to a shaft. The method may include inserting an anvil into a slot defined by the shaft. The method may include engaging a first end of a tip with a surface of the anvil. The method may include constraining the tip with a sleeve.

### BRIEF DESCRIPTION OF THE DRAWINGS

The features and advantages of the invention will be more fully disclosed in, or rendered obvious by, the following detailed exemplary descriptions of embodiments. The detailed descriptions of these exemplary embodiments are to be considered together with the accompanying drawings, wherein like numbers refer to like parts and further wherein:

FIG. 1 illustrates a side view of a first surgical driver in accordance with some embodiments;

FIG. 2 illustrates an enlarged side view of a first surgical driver as illustrated in FIG. 1 in accordance with some embodiments;

FIG. 3 illustrates an isometric view of a first surgical driver in accordance with some embodiments;

FIG. 4 illustrates a cross-sectional view of a first surgical driver along axis 4-4 illustrated in FIG. 3 in accordance with some embodiments;

FIG. 5 illustrates an enlarged cross-sectional view of a first surgical driver as illustrated in FIG. 4 in accordance with some embodiments;

FIG. 6 illustrates a side view of a handle of a first surgical driver in accordance with some embodiments;

FIG. 7 illustrates an enlarged side view of a shaft of a first surgical driver in accordance with some embodiments;

FIG. 8 illustrates an isometric view of an anvil of a first surgical driver in accordance with some embodiments;

FIG. 9 illustrates an isometric view of a tip of a first surgical driver in accordance with some embodiments;

FIG. 10 illustrates a side view of a sleeve of a first surgical driver in accordance with some embodiments;

FIG. 11 illustrates an exploded view of a first surgical driver in accordance with some embodiments;

FIG. 12 illustrates an enlarged exploded view of a first surgical driver in accordance with some embodiments;

FIG. 13 illustrates a side view of a first surgical driver along detail A illustrated in FIG. 2 in accordance with some embodiments;

FIG. 14 illustrates a cross-sectional view of a first surgical driver along axis 14-14 illustrated in FIG. 13 in accordance with some embodiments;

FIG. 15 illustrates an exploded view of a shaft and a tip of a first surgical driver in accordance with some embodiments;

FIG. 16 illustrates an isometric view of a shaft and a tip of a first surgical driver illustrated in FIG. 15 with the shaft and tip coupled together in accordance with some embodiments;

FIG. 17 illustrates a side view of a second surgical driver in accordance with some embodiments;

FIG. 18 illustrates a top view of a second surgical driver in accordance with some embodiments;

FIG. 19 illustrates an isometric view of a second surgical driver in accordance with some embodiments;

FIG. 20 illustrates a cross-sectional view of a second surgical driver along axis 20-20 illustrated in FIG. 19 in accordance with some embodiments;

FIG. 21 illustrates an enlarged cross-sectional view of a second surgical driver illustrated in FIG. 20 in accordance with some embodiments;

FIG. 22 illustrates a side view of a handle of a second surgical driver in accordance with some embodiments;

FIG. 23 illustrates a side view of a shaft of a second surgical driver in accordance with some embodiments;

FIG. 24 illustrates an isometric view of an anvil of a second surgical driver in accordance with some embodiments;

FIG. 25 illustrates an isometric view of a tip of a second surgical driver in accordance with some embodiments;

FIG. 26 illustrates a side view of a sleeve of a second surgical driver in accordance with some embodiments;

FIG. 27 illustrates a side view of a rod of a second surgical driver in accordance with some embodiments;

FIG. 28 illustrates an exploded view of a second surgical driver in accordance with some embodiments;

FIG. 29 illustrates an enlarged exploded view of a second surgical driver illustrated in FIG. 28 in accordance with some embodiments;

FIG. 30 illustrates a cross-sectional view of a second surgical driver along detail B illustrated in FIG. 20 in accordance with some embodiments;

FIG. 31 illustrates a second cross-sectional view of a second surgical driver along detail B illustrated in FIG. 20 in accordance with some embodiments; and

FIG. 32 illustrates an exemplary method of assembling a surgical driver in accordance with some embodiments.

### DETAILED DESCRIPTION

This description of the exemplary embodiments is intended to be read in connection with the accompanying drawings, which are to be considered part of the entire written description. It should be understood, however, that the present disclosure is not intended to be limited to the particular forms disclosed and that the drawings are not necessarily shown to scale. Rather, the present disclosure covers all modifications, equivalents, and alternatives that fall within the spirit and scope of these exemplary embodiments. In the description, relative terms such as “lower,” “upper,” “horizontal,” “vertical,” “above,” “below,” “up,” “down,” “top,” and “bottom” as well as derivatives thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The terms “couple,” “coupled,” “operatively coupled,” “operatively connected,” and the like should be broadly understood to refer to connecting devices or components together either mechanically, or otherwise, such that the connection allows the pertinent devices or components to operate with each other as intended by virtue of that relationship.

The drivers, systems, kits, and methods being disclosed allow a surgeon to transmit off-axis torque to a surgical tool, instrument, or implant during a procedure at a constant velocity and at a variable angle. Some advantages of the drivers disclosed herein include: (1) a 1:1 input-to-output torque ratio, which is useful in applications where torque must be maintained; (2) a smoother drive for the user; (3) reduced friction due to rolling bearing balls; (4) a cageless design, making the driver more compact; and (5) a pin-less design, which eliminates the need for through holes, thin walls, and welds, making the driver relatively stronger than other drivers.

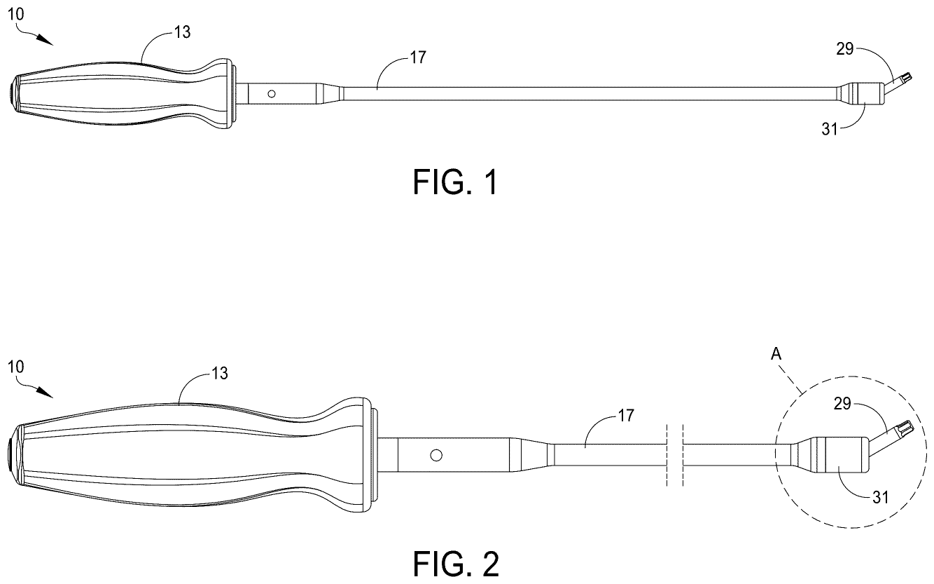

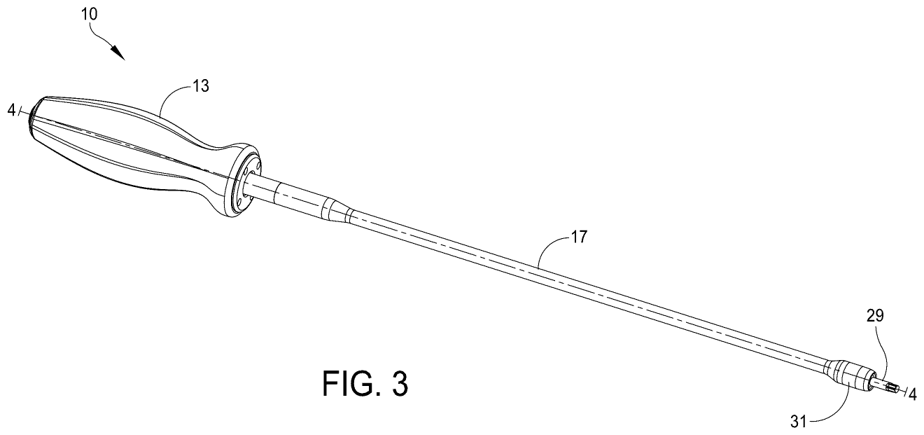

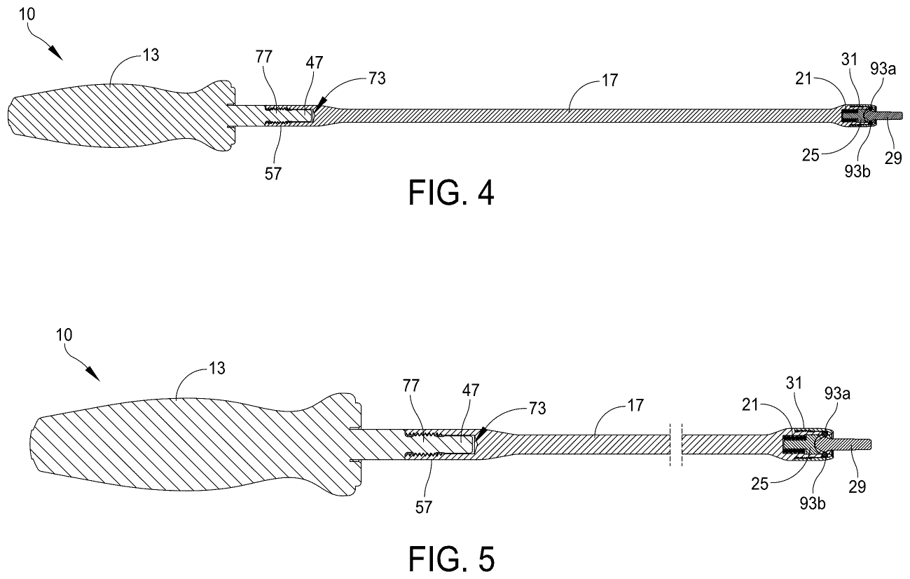

Turning now to the drawings, FIGS. 1-3 illustrate side and isometric views of a first surgical driver 10 and FIGS. 4-5 illustrate cross-sectional views of surgical driver 10 along axis 4-4 illustrated in FIG. 3 in accordance with some embodiments. Surgical driver 10 has a handle 13, a shaft 17, a biasing member 21 and an anvil 25 as best illustrated in FIGS. 4-5, a tip 29, and a sleeve 31.

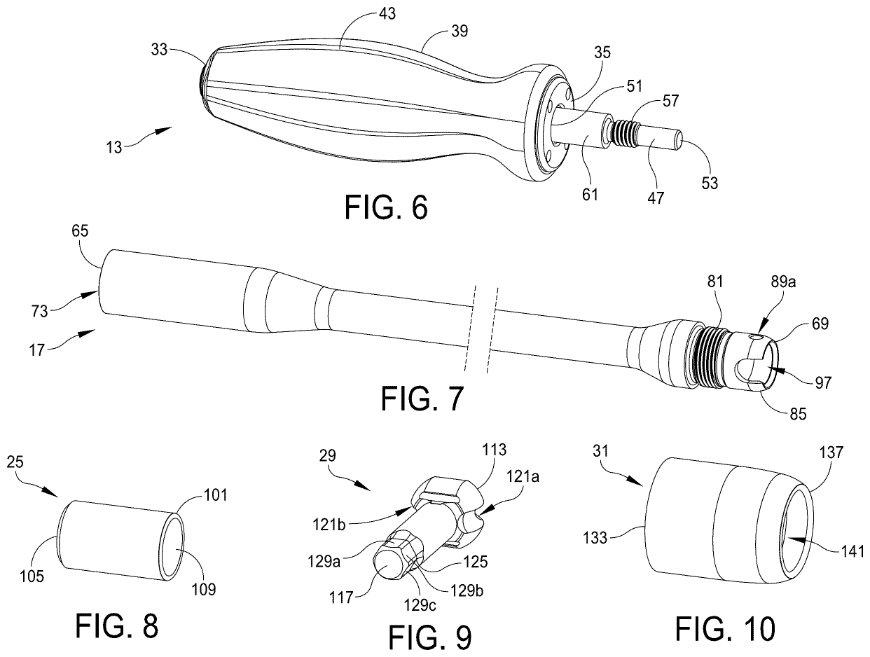

FIG. 6 illustrates a side view of handle 13 of surgical driver 10 in accordance with some embodiments. Handle 13 extends between a first end 33 and a second end 35. Handle 13 defines a grip 39 disposed between first end 33 and second end 35. Grip 39 is sized and configured to be grasped by a surgeon. In some embodiments, grip 39 includes a grip feature 43, such as a non-skid surface, a plurality of ridges, a wrap, etc. to better facilitate grasping handle 13 by a surgeon.

Handle 13 includes a securing member 47 that extends between a first end 51 and a second end 53. First end 51 of securing member 47 is coupled to second end 35 of handle 13. In some embodiments, securing member 47 is fixedly or removably coupled to handle 13. In some embodiments, securing member 47 is formed integrally with handle 13. Securing member 47 has a securing portion 57 near second end 53 and a torque feature 61 near first end 51. Securing portion 57 may have one or more surface features, such as a threaded portion, sized and configured to couple handle 13 and shaft 17. For example, securing portion 57 may have threads that are configured to engage respective threads on shaft 17. It will be appreciated that securing member 47 and shaft 17 may be coupled together with different or additional features, such as a medical-grade adhesive.

Torque feature 61 allows handle 13 to input a predetermined torque before slipping such that the predetermined torque is not exceeded. For example, torque feature 61 may be similar to a torque wrench. In some embodiments, the predetermined torque setting may be 0.1-1 N·m. In some embodiments, the predetermined torque setting may be about 0.6 N·m. For example, the driver 10 may be used with a 0.6 N·m torque handle to rotate one or more cams of Stryker Corporation's Monterey AL interbody implant, preventing the fasteners coupled to the implant from backing out. It will be appreciated that the predetermined torque setting of torque feature 61 may be set to any suitable torque setting depending on the application and use of surgical driver 10.

Handle 13 may be any suitable material, such as a metal, metal alloy, or plastic. In some embodiments, handle 13 may be formed from a medical-grade material that is capable of being 3D printed (e.g., additively manufactured), such as ABS (acrylonitrile butadiene styrene), PLA (polylactic acid), PETG (polyethylene terephthalate glycol), nylon, TPU (thermoplastic polyurethane), resin, and other suitable thermoplastics and thermosetting plastics. However, handle 13 may be formed from other materials, including metals, ceramics, and other materials that are suitable for use in surgery as will be understood by one of ordinary skill in the art. In some embodiments, handle 13 may be machined and/or formed using an additive manufacturing process, such as electron beam melting (EBM) or direct metal laser sintering (DMLS), to list only a few possibilities.

It will be appreciated that portions of handle 13 (e.g., first end 33, second end 35, grip 39, and securing member 47) may be discrete parts coupled together or are formed integrally together. In some embodiments, portions of handle 13 (e.g., first end 33, second end 35, grip 39, and securing member 47) are the same material. In some embodiments, portions of handle 13 (e.g., first end 33, second end 35, grip 39, and securing member 47) are a different material.

FIG. 7 illustrates an enlarged side view of shaft 17 of surgical driver 10 in accordance with some embodiments. Shaft 17 extends between a first end 65 and a second end 69. Shaft 17 defines an aperture 73 at first end 65. As best illustrated in FIG. 5, shaft 17 defines a securing feature 77 within aperture 73. Securing feature 77 may have one or more surface features, such as a threaded portion, sized and configured to couple handle 13 and shaft 17. For example, securing feature 77 may have threads that are configured to engage respective threads of securing portion 57. It will be appreciated that securing member 47 and shaft 17 may be coupled together with different or additional features, such as a medical-grade adhesive.

Referring back to FIG. 7, shaft 17 defines a second securing feature 81 and a retaining feature 85 near second end 69. Second securing feature 81 may have one or more surface features, such as a threaded portion, sized and configured to couple shaft 17 and sleeve 31. For example, second securing feature 81 may have threads that are configured to engage respective threads of sleeve 31. It will be appreciated that shaft 17 and sleeve 31 may be coupled together with different or additional features.

Retaining feature 85 defines one or more holes 89a-b. As best illustrated in FIG. 5, holes 89a-b are sized and configured to receive a respective bearing 93a-b. Bearings 93a-b are configured to retain tip 29 and facilitate movement of tip 29 relative to a longitudinal axis of shaft 17 as will be discussed in more detail below. In some embodiments, bearings 93a-b may be ball bearings. Referring back to FIG. 7, shaft 17 also defines a slot 97 at second end 69. Slot 97 is sized and configured to receive biasing member 21, anvil 25, and a portion of tip 29.

Shaft 17 may be any suitable material, such as a metal, metal alloy, or plastic. In some embodiments, shaft 17 may be formed from a medical-grade material that is capable of being 3D printed (e.g., additively manufactured), such as ABS (acrylonitrile butadiene styrene), PLA (polylactic acid), PETG (polyethylene terephthalate glycol), nylon, TPU (thermoplastic polyurethane), resin, and other suitable thermoplastics and thermosetting plastics. However, shaft 17 may be formed from other materials, including metals, ceramics, and other materials that are suitable for use in surgery as will be understood by one of ordinary skill in the art. In some embodiments, shaft 17 may be machined and/or formed using an additive manufacturing process, such as electron beam melting (EBM) or direct metal laser sintering (DMLS), to list only a few possibilities.

It will be appreciated that portions of shaft 17 (e.g., first end 65, second end 69, securing features 77, 81, and retaining feature 85) may be discrete parts coupled together or are formed integrally together. In some embodiments, portions of shaft 17 (e.g., first end 65, second end 69, securing features 77, 81, and retaining feature 85) are the same material. In some embodiments, portions of shaft 17 (e.g., first end 65, second end 69, securing features 77, 81, and retaining feature 85) are a different material.

As best illustrated in FIG. 14, biasing member 21 is sized and configured to be received within slot 97 of shaft 17. Biasing member 21 is sized and configured to bias anvil 25 against shaft 17 as will be discussed in more detail below. In some embodiments, biasing member 21 is one or more springs. In some embodiments, biasing member 21 includes nested springs with opposite directions of wind to increase the biasing force. In some embodiments, biasing member 21 is a metal, metal alloy, plastic, or some other suitable material as discussed herein.

FIG. 8 illustrates an isometric view of anvil 25 of surgical driver 10 in accordance with some embodiments. Anvil 25 is sized and configured to be received within slot 97 of shaft 17. Anvil 25 extends between a first end 101 and a second end 105. First end 101 of anvil 25 defines a surface 109. Surface 109 is sized and configured to receive a portion of tip 29 as will be discussed in more detail below. As best seen in FIG. 14, surface 109 is curved according to some embodiments. Second end 105 of anvil 25 engages biasing member 21 such that anvil 25 is biased against shaft 17 when disposed within slot 97.

Anvil 25 may be any suitable material, such as a metal, metal alloy, or plastic. In some embodiments, anvil 25 may be formed from a medical-grade material that is capable of being 3D printed (e.g., additively manufactured), such as ABS (acrylonitrile butadiene styrene), PLA (polylactic acid), PETG (polyethylene terephthalate glycol), nylon, TPU (thermoplastic polyurethane), resin, and other suitable thermoplastics and thermosetting plastics. However, anvil 25 may be formed from other materials, including metals, ceramics, and other materials that are suitable for use in surgery as will be understood by one of ordinary skill in the art. In some embodiments, anvil 25 may be machined and/or formed using an additive manufacturing process, such as electron beam melting (EBM) or direct metal laser sintering (DMLS), to list only a few possibilities.

FIG. 9 illustrates an isometric view of tip 29 of surgical driver 10 in accordance with some embodiments. Tip 29 extends between a first end 113 and a second end 117. First end 113 is sized and configured to be received within slot 97 of shaft 17 and engage surface 109 of anvil 25 when assembled. Tip 29 defines one or more slots 121a-b. Slots 121a-b are sized and configured receive respective bearings 93a-b. With bearings 93a-b disposed within holes 89a-b and slots 121a-b, tip 29 is coupled to shaft 17. In some embodiments, slots 121a-b are circular in cross section and concentric with the ball-and-socket joint. In some embodiments, slots 121a-b may be machined into shaft 17.

Second end 117 of tip 29 defines an operating end 125. Operating end 125 is sized and configured to receive a surgical tool, instrument, or implant. In some embodiments, operating end 125 defines one or more surface features 129a-c. For example, surface features 129a-c may be threads and configured to receive respective threads of a surgical tool, instrument, or implant. In other embodiments, surface feature 129a-c define a drill bit or fastener head (e.g., flat head bit, Philips-head bit, Allen-drive bit, etc.). In further embodiments, operating end 125 defines an aperture configured to receive a surgical tool, instrument, or implant. For example, operating end 125 may be sized and configured to receive a drill bit or fastener head (e.g., flat head bit, Philips-head bit, Allen-drive bit, etc.). In some embodiments, operating end 125 may be formed integrally with a surgical tool or instrument such that the surgical tool or instrument is disposed on second end 117 of tip 29.

Tip 29 may be any suitable material, such as a metal, metal alloy, or plastic. In some embodiments, tip 29 may be formed from a medical-grade material that is capable of being 3D printed (e.g., additively manufactured), such as ABS (acrylonitrile butadiene styrene), PLA (polylactic acid), PETG (polyethylene terephthalate glycol), nylon, TPU (thermoplastic polyurethane), resin, and other suitable thermoplastics and thermosetting plastics. However, tip 29 may be formed from other materials, including metals, ceramics, and other materials that are suitable for use in surgery as will be understood by one of ordinary skill in the art. In some embodiments, tip 29 may be machined and/or formed using an additive manufacturing process, such as electron beam melting (EBM) or direct metal laser sintering (DMLS), to list only a few possibilities.

FIG. 10 illustrates a side view of sleeve 31 of surgical driver 10 in accordance with some embodiments. Sleeve 31 extends between a first end 133 and a second end 137. Sleeve 31 defines a void 141 that extends between first end 133 and second end 137. As best seen in FIG. 14, sleeve 31 defines a securing feature 145 within void 141. Securing feature 145 may have one or more surface features, such as a threaded portion, sized and configured to couple shaft 17 and sleeve 31. For example, securing feature 145 may have threads that are configured to engage respective threads of second securing feature 81. It will be appreciated that shaft 17 and sleeve 31 may be coupled together with different or additional features.

Sleeve 31 may be any suitable material, such as a metal, metal alloy, or plastic. In some embodiments, sleeve 31 may be formed from a medical-grade material that is capable of being 3D printed (e.g., additively manufactured), such as ABS (acrylonitrile butadiene styrene), PLA (polylactic acid), PETG (polyethylene terephthalate glycol), nylon, TPU (thermoplastic polyurethane), resin, and other suitable thermoplastics and thermosetting plastics. However, sleeve 31 may be formed from other materials, including metals, ceramics, and other materials that are suitable for use in surgery as will be understood by one of ordinary skill in the art. In some embodiments, sleeve 31 may be machined and/or formed using an additive manufacturing process, such as electron beam melting (EBM) or direct metal laser sintering (DMLS), to list only a few possibilities.

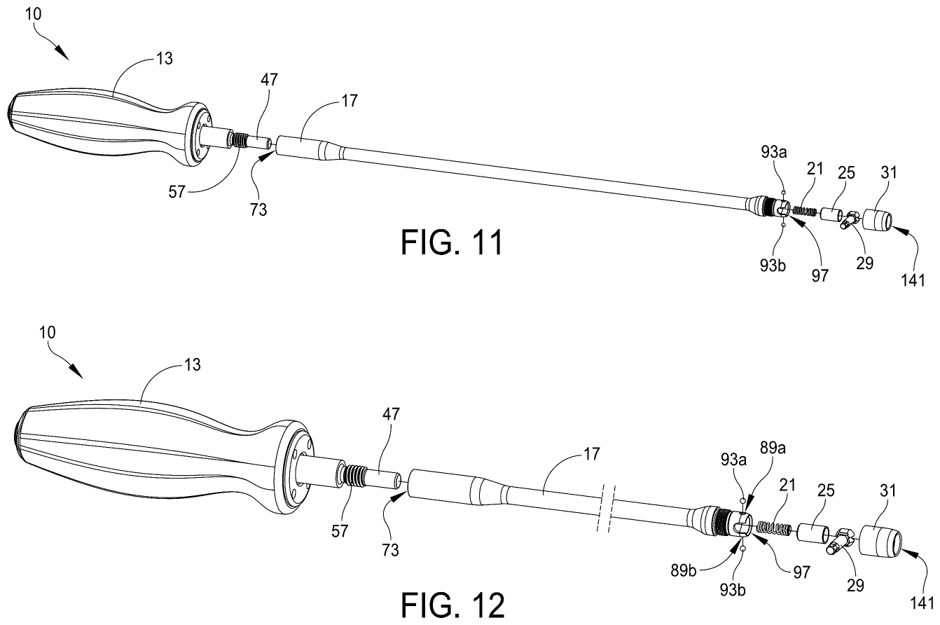

FIGS. 11-12 illustrate exploded views of surgical driver 10 in accordance with some embodiments. Surgical driver 10 may be sold already assembled, as illustrated in FIGS. 1-2, or come as a kit for a user to assemble as illustrated in FIGS. 11-12. It will be appreciated that surgical driver 10 may be disassembled and reassembled by a user for cleaning, maintenance, repair, or to exchange one or more parts. For example, a user may desire a different tip 29 with a different operating end 125 as discussed herein.

Surgical driver 10 is assembled by inserting securing member 47 of handle 13 into aperture 73 of shaft 17. Securing portion 57 of securing member 47 and securing feature 77 of shaft 17 engage and couple handle 13 to shaft 17. For example, securing portion 57 may be threaded and securing feature 77 is also threaded with respective threads such that securing portion 57 and securing feature 77 are removably coupled by the threads.

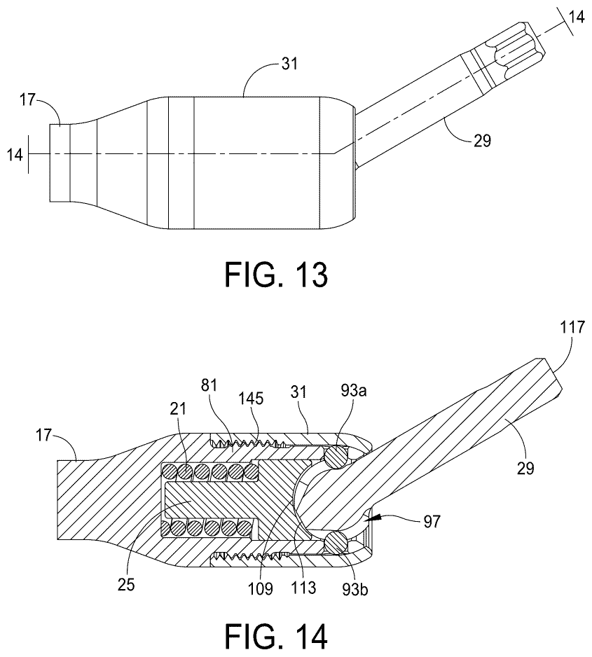

FIG. 13 illustrates a side view of surgical driver 10 along detail A illustrated in FIG. 2 and FIG. 14 illustrates a cross-sectional view of surgical driver 10 along axis 14-14 illustrated in FIG. 13 in accordance with some embodiments. Biasing member 21 is inserted into slot 97 of shaft 17. Anvil 25 is also inserted into slot 97 over biasing member 21 such that anvil 25 is biased against shaft 17 with biasing member 21. As best illustrated in FIG. 14, tip 29 is inserted into slot 97 such that first end 113 of tip 29 engages surface 109 of anvil 25.

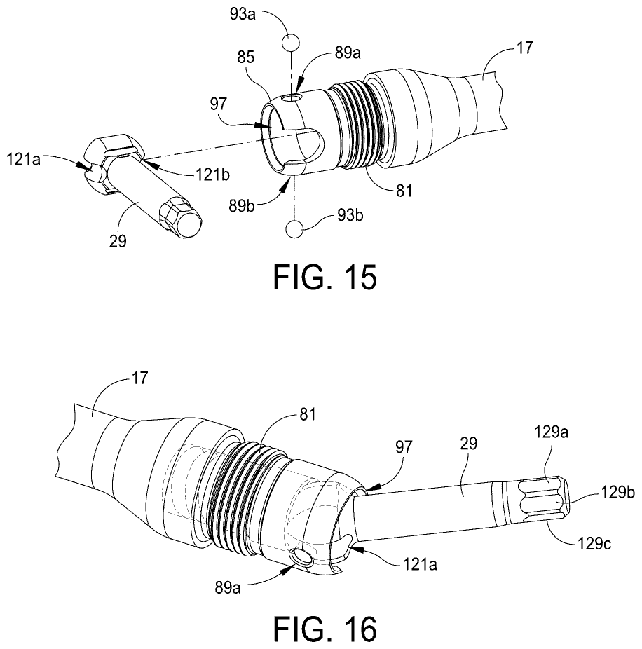

FIG. 15 illustrates an exploded view of shaft 17 and tip 29 of surgical driver 10 and FIG. 16 illustrates an isometric view of shaft 17 and tip 29 of surgical driver 10 illustrated in FIG. 15 with shaft 17 and tip 29 coupled together in accordance with some embodiments. Bearings 93a-b are then inserted into holes 89a-b of retaining feature 85 and into slots 121a-b of tip 29. Referring back to FIG. 14, sleeve 31 is then slid over tip 29 and shaft 17 such that tip 29 and shaft 17 are disposed within void 141. With sleeve 31 installed, securing feature 145 of sleeve 31 engages second securing feature 81 of shaft 17 to couple sleeve 31 and shaft 17 together. For example, securing feature 145 and second securing feature 81 are threaded with respective threads such that securing feature 145 and second securing feature 81 are removably coupled by the threads.

Sleeve 31 constrains tip 29 within slot 97. Movement of tip 29 is facilitated by anvil 25 and bearings 93a-b such that a user can orient operating end 125 from a first angle to a second angle (e.g., from along the longitudinal axis of surgical driver 10 illustrated in FIG. 3 to some angle as illustrated in FIGS. 1-2). Biasing member 21 also prevents tip 29 from moving without user input. For example, the force of biasing member 21 against anvil 25 keeps operating end 125 oriented at the angle set by the user. This feature allows the user to control the position and orientation of the tip 29. The intersection angle between the input shaft (i.e., shaft 17) and output shaft (i.e., tip 29) is also variable within a set upper and lower bounds. For example, the intersection angle may be set to ±30°, but it will be appreciated that this value may be increased or decreased as desired.

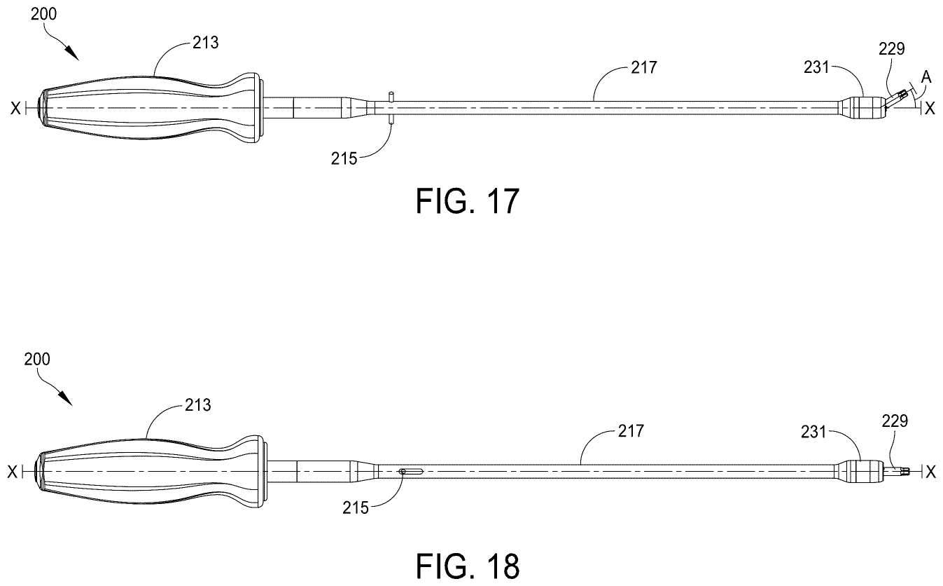

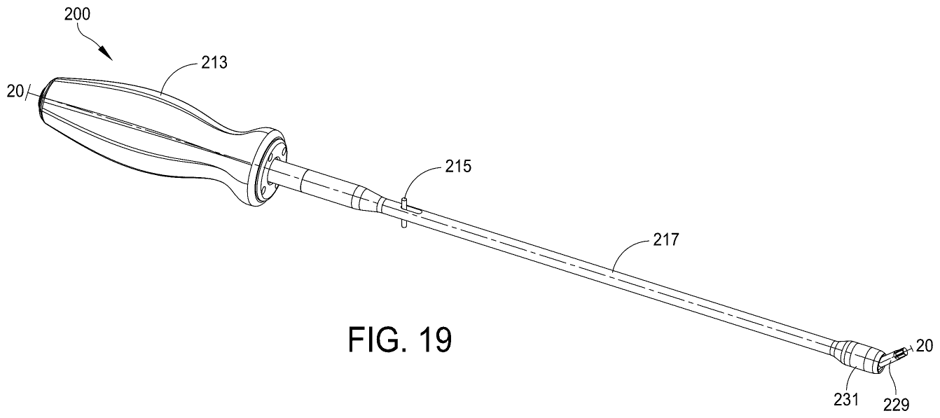

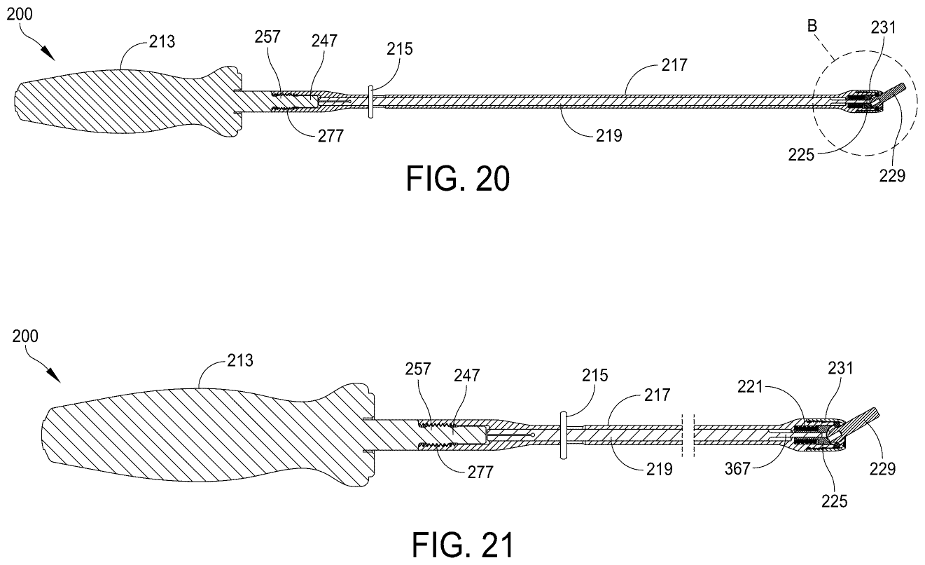

FIGS. 17-19 illustrate side, top, and isometric views of a second surgical driver 200 and FIGS. 20-21 illustrate cross-sectional views of surgical driver 200 in accordance with some embodiments. Surgical driver 200 may be similar to surgical driver 10 as discussed above. Surgical driver 200 has a handle 213, a pin 215, a shaft 217, a rod 219 as best illustrated in FIGS. 20-21, a biasing member 221 and an anvil 225 as best illustrated in FIG. 21, a tip 229, and a sleeve 231.

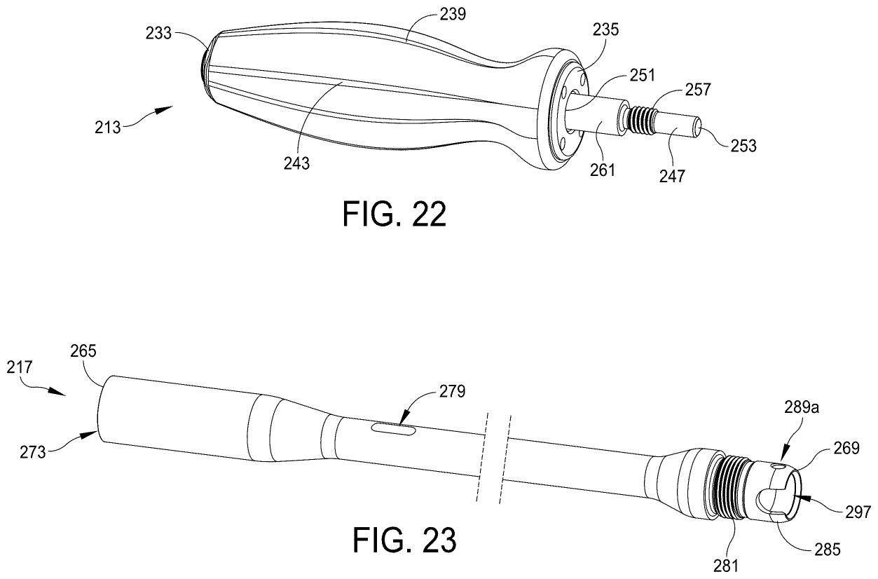

FIG. 22 illustrates a side view of handle 213 of surgical driver 200 in accordance with some embodiments. Handle 213 extends between a first end 233 and a second end 235. Handle 213 defines a grip 239 disposed between first end 233 and second end 235. Grip 239 is sized and configured to be grasped by a surgeon. In some embodiments, grip 239 includes a grip feature 243, such as a non-skid surface, a plurality of ridges, a wrap, etc. to better facilitate grasping handle 213 by a surgeon.

Handle 213 includes a securing member 247. Securing member 247 extends between a first end 251 and a second end 253. First end 251 of securing member 247 is coupled to second end 235 of handle 213. In some embodiments, securing member 247 is fixedly or removably coupled to handle 213. In some embodiments, securing member 247 is formed integrally with handle 213. Securing member 247 has a securing portion 257 near second end 253 and a torque feature 261 near first end 251. Securing portion 257 may have one or more surface features, such as a threaded portion, sized and configured to couple handle 213 and shaft 217. For example, securing portion 257 may have threads that are configured to engage respective threads on shaft 217. It will be appreciated that securing member 247 and shaft 217 may be coupled together with different or additional features, such as a medical-grade adhesive.

Torque feature 261 allows handle 213 to input a predetermined torque before slipping such that the predetermined torque is not exceeded. For example, torque feature 261 may be similar to a torque wrench. In some embodiments, the predetermined torque setting may be 0.1-1 N·m. In some embodiments, the predetermined torque setting may be about 0.6 N·m. For example, the surgical driver 200 may be used with a 0.6 N·m torque handle to rotate one or more cams of Stryker Corporation's Monterey AL interbody implant, preventing the fasteners coupled to the implant from backing out. It will be appreciated that the predetermined torque setting of torque feature 261 may be set to any suitable torque setting depending on the application and use of surgical driver 200.

Handle 213 may be any suitable material, such as a metal, metal alloy, or plastic. In some embodiments, handle 213 may be formed from a medical-grade material that is capable of being 3D printed (e.g., additively manufactured), such as ABS (acrylonitrile butadiene styrene), PLA (polylactic acid), PETG (polyethylene terephthalate glycol), nylon, TPU (thermoplastic polyurethane), resin, and other suitable thermoplastics and thermosetting plastics. However, handle 213 may be formed from other materials, including metals, ceramics, and other materials that are suitable for use in surgery as will be understood by one of ordinary skill in the art. In some embodiments, handle 213 may be machined and/or formed using an additive manufacturing process, such as electron beam melting (EBM) or direct metal laser sintering (DMLS), to list only a few possibilities.

It will be appreciated that portions of handle 213 (e.g., first end 233, second end 235, grip 239, and securing member 247) may be discrete parts coupled together or are formed integrally together. In some embodiments, portions of handle 213 (e.g., first end 233, second end 235, grip 239, and securing member 247) are the same material. In some embodiments, portions of handle 213 (e.g., first end 233, second end 235, grip 239, and securing member 247) are a different material.

FIG. 23 illustrates a side view of shaft 217 of surgical driver 200 in accordance with some embodiments. Shaft 217 extends between a first end 265 and a second end 269. Shaft 217 defines an aperture 273 that extends between first end 265 and second end 269. As best illustrated in FIG. 21, shaft 217 defines a securing feature 277 within aperture 273 near first end 265. Securing feature 277 may have one or more surface features, such as a threaded portion, sized and configured to couple handle 213 and shaft 217. For example, securing feature 277 may have threads that are configured to engage respective threads of securing portion 257. It will be appreciated that securing member 247 and shaft 217 may be coupled together with different or additional features, such as a medical-grade adhesive.

Aperture 273 is sized and configured to receive rod 219 therein such that rod 219 extends between first end 265 and second end 269 of shaft 217. Referring back to FIG. 23, shaft 217 defines a slot 279 near first end 265 that extends through shaft 217. Slot 279 is sized and configured to receive pin 215.

Shaft 217 defines a second securing feature 281 and a retaining feature 285 near second end 269. Second securing feature 281 may have one or more surface features, such as a threaded portion, sized and configured to couple shaft 217 and sleeve 231. For example, second securing feature 281 may have threads that are configured to engage respective threads of sleeve 231. It will be appreciated that shaft 217 and sleeve 231 may be coupled together with different or additional features.

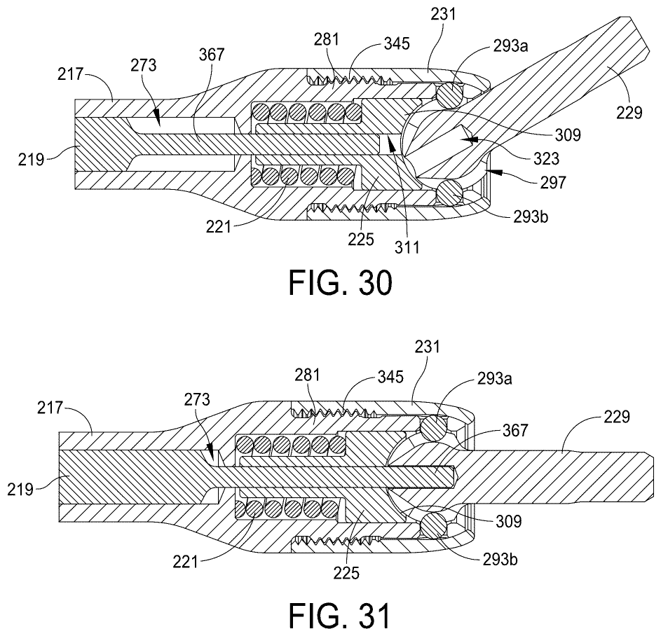

Retaining feature 285 defines one or more holes 289a-b. Holes 289a-b are sized and configured to receive a respective bearing 293a-b, as best illustrated in FIGS. 30-31. Bearings 293a-b are configured to retain tip 229 and facilitate movement of tip 229 relative to a longitudinal axis of shaft 217 as will be discussed in more detail below. In some embodiments, bearings 293a-b may be ball bearings. Shaft 217 also defines a slot 297 at second end 269. Slot 297 is sized and configured to receive a portion of rod 219, biasing member 221, anvil 225, and a portion of tip 229.

Shaft 217 may be any suitable material, such as a metal, metal alloy, or plastic. In some embodiments, shaft 217 may be formed from a medical-grade material that is capable of being 3D printed (e.g., additively manufactured), such as ABS (acrylonitrile butadiene styrene), PLA (polylactic acid), PETG (polyethylene terephthalate glycol), nylon, TPU (thermoplastic polyurethane), resin, and other suitable thermoplastics and thermosetting plastics. However, shaft 217 may be formed from other materials, including metals, ceramics, and other materials that are suitable for use in surgery as will be understood by one of ordinary skill in the art. In some embodiments, shaft 217 may be machined and/or formed using an additive manufacturing process, such as electron beam melting (EBM) or direct metal laser sintering (DMLS), to list only a few possibilities.

It will be appreciated that portions of shaft 217 (e.g., first end 265, second end 269, securing features 277, 281, and retaining feature 285) may be discrete parts coupled together or are formed integrally together. In some embodiments, portions of shaft 217 (e.g., first end 265, second end 269, securing features 277, 281, and retaining feature 285) are the same material. In some embodiments, portions of shaft 217 (e.g., first end 265, second end 269, securing features 277, 281, and retaining feature 285) are a different material.

As best illustrated in FIGS. 30-31, biasing member 221 is sized and configured to be received within slot 297. Biasing member 221 is sized and configured to bias anvil 225 against shaft 217 as will be discussed in more detail below. In some embodiments, biasing member 221 is one or more springs. In some embodiments, biasing member 221 includes nested springs with opposite directions of wind to increase the biasing force. In some embodiments, biasing member 221 is a metal, metal alloy, plastic, or some other suitable material as discussed herein.

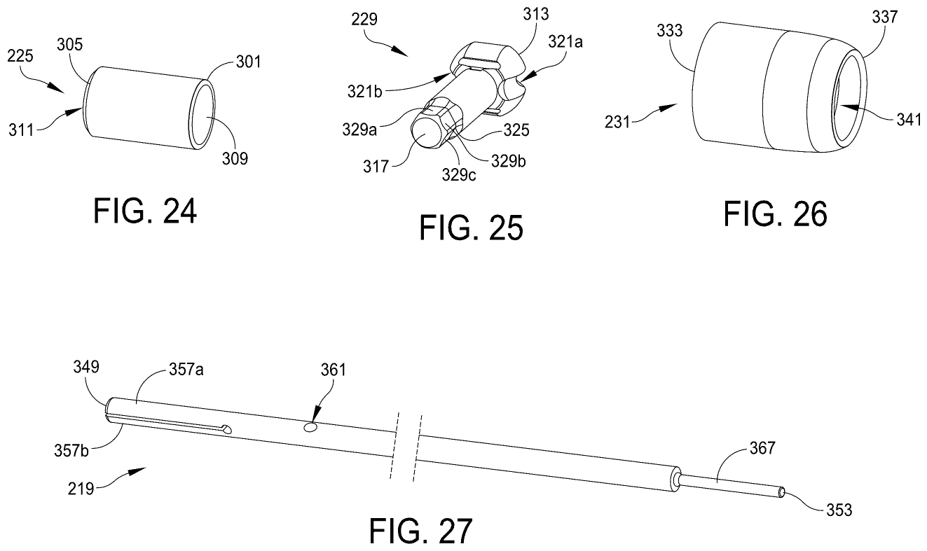

FIG. 24 illustrates an isometric view of anvil 225 of surgical driver 200 in accordance with some embodiments. Anvil 225 is sized and configured to be received within slot 297. Anvil 225 extends between a first end 301 and a second end 305. First end 301 of anvil 225 defines a surface 309. Surface 309 is sized and configured to receive a portion of tip 229 as will be discussed in more detail below. As best illustrated in FIGS. 30-31, surface 309 is curved according to some embodiments. As best illustrated in FIGS. 30-31, second end 305 of anvil 225 defines a void 311 that extends through anvil 225 from first end 301 to second end 305. Void 311 is sized and configured to receive a portion of rod 219 as will be discussed in more detail below. Second end 305 of anvil 225 engages biasing member 221 such that anvil 225 is biased against shaft 217 when disposed within slot 297.

Anvil 225 may be any suitable material, such as a metal, metal alloy, or plastic. In some embodiments, anvil 225 may be formed from a medical-grade material that is capable of being 3D printed (e.g., additively manufactured), such as ABS (acrylonitrile butadiene styrene), PLA (polylactic acid), PETG (polyethylene terephthalate glycol), nylon, TPU (thermoplastic polyurethane), resin, and other suitable thermoplastics and thermosetting plastics. However, anvil 225 may be formed from other materials, including metals, ceramics, and other materials that are suitable for use in surgery as will be understood by one of ordinary skill in the art. In some embodiments, anvil 225 may be machined and/or formed using an additive manufacturing process, such as electron beam melting (EBM) or direct metal laser sintering (DMLS), to list only a few possibilities.

FIG. 25 illustrates an isometric view of tip 229 of surgical driver 200 in accordance with some embodiments. Tip 229 extends between a first end 313 and a second end 317. First end 313 is sized and configured to be received within slot 297 of shaft 217 and engage surface 309 of anvil 225 when assembled. Tip 229 defines one or more slots 321a-b. Slots 321a-b are sized and configured receive respective bearings 293a-b. With bearings 293a-b disposed within holes 289a-b and slots 321a-b, tip 229 is coupled to shaft 217. As best illustrated in FIGS. 30-31, tip 229 defines a hole 323 at first end 313. Hole 323 is sized and configured to receive a portion of rod 219 that extends through anvil 225 as discussed in more detail below.

Second end 317 of tip 229 defines an operating end 325. Operating end 325 is sized and configured to receive a surgical tool, instrument, or implant. In some embodiments, operating end 325 defines one or more surface features 329a-c. For example, surface features 329a-c may be threads and configured to receive respective threads of a surgical tool, instrument, or implant. In other embodiments, surface feature 329a-c define a drill bit or fastener head (e.g., flat head bit, Philips-head bit, Allen-drive bit, etc.). In further embodiments, operating end 325 defines an aperture configured to receive a surgical tool, instrument, or implant. For example, operating end 325 may be sized and configured to receive a drill bit or fastener head (e.g., flat head bit, Philips-head bit, Allen-drive bit, etc.). In some embodiments, operating end 325 may be formed integrally with a surgical tool or instrument such that the surgical tool or instrument is disposed on second end 317 of tip 229.

Tip 229 may be any suitable material, such as a metal, metal alloy, or plastic. In some embodiments, tip 229 may be formed from a medical-grade material that is capable of being 3D printed (e.g., additively manufactured), such as ABS (acrylonitrile butadiene styrene), PLA (polylactic acid), PETG (polyethylene terephthalate glycol), nylon, TPU (thermoplastic polyurethane), resin, and other suitable thermoplastics and thermosetting plastics. However, tip 229 may be formed from other materials, including metals, ceramics, and other materials that are suitable for use in surgery as will be understood by one of ordinary skill in the art. In some embodiments, tip 229 may be machined and/or formed using an additive manufacturing process, such as electron beam melting (EBM) or direct metal laser sintering (DMLS), to list only a few possibilities.

FIG. 26 illustrates a side view of sleeve 231 of surgical driver 200 in accordance with some embodiments. Sleeve 231 extends between a first end 333 and a second end 337. Sleeve 231 defines a void 341 that extends from first end 333 to second end 337. As best illustrated in FIGS. 30-31, sleeve 231 defines a securing feature 345 within void 341. Securing feature 345 may have one or more surface features, such as a threaded portion, sized and configured to couple shaft 217 and sleeve 231 together. For example, securing feature 345 may have threads that are configured to engage respective threads of second securing feature 281. It will be appreciated that shaft 217 and sleeve 231 may be coupled together with different or additional features.

Sleeve 231 may be any suitable material, such as a metal, metal alloy, or plastic. In some embodiments, sleeve 231 may be formed from a medical-grade material that is capable of being 3D printed (e.g., additively manufactured), such as ABS (acrylonitrile butadiene styrene), PLA (polylactic acid), PETG (polyethylene terephthalate glycol), nylon, TPU (thermoplastic polyurethane), resin, and other suitable thermoplastics and thermosetting plastics. However, sleeve 231 may be formed from other materials, including metals, ceramics, and other materials that are suitable for use in surgery as will be understood by one of ordinary skill in the art. In some embodiments, sleeve 231 may be machined and/or formed using an additive manufacturing process, such as electron beam melting (EBM) or direct metal laser sintering (DMLS), to list only a few possibilities.

FIG. 27 illustrates a side view of rod 219 of surgical driver 200 in accordance with some embodiments. Rod 219 extends between a first end 349 and a second end 353. First end 349 defines a pair of wedges 357a-b. Rod 219 defines an aperture 361 near first end 349. Wedges 357a-b are sized and configured to receive pin 215 between them such that the wedges 357a-b direct pin 215 into aperture 361. For example, movement of pin 215 by a user toward second end 269 of shaft 217 pushes pin 215 between wedges 357a-b until pin 215 reaches aperture 361. With pin 215 disposed within aperture 361, wedges 357a-b hold pin 215 in position. Conversely, movement of pin 215 by a user toward handle 213 moves pin 215 out of aperture 361 and back out of wedges 357a-b.

Second end 353 of rod 219 defines a fixation portion 367. Fixation portion 367 is sized and configured to be received within void 311 of anvil 225 and within hole 323 of tip 229 to lock tip 229 along the longitudinal axis of surgical driver 200. For example, operation of pin 215 as discussed above moves fixation portion 367 in and out of hole 323 to fixate tip 229 or free tip 229 to move.

Rod 219 may be any suitable material, such as a metal, metal alloy, or plastic. In some embodiments, rod 219 may be formed from a medical-grade material that is capable of being 3D printed (e.g., additively manufactured), such as ABS (acrylonitrile butadiene styrene), PLA (polylactic acid), PETG (polyethylene terephthalate glycol), nylon, TPU (thermoplastic polyurethane), resin, and other suitable thermoplastics and thermosetting plastics. However, rod 219 may be formed from other materials, including metals, ceramics, and other materials that are suitable for use in surgery as will be understood by one of ordinary skill in the art. In some embodiments, rod 219 may be machined and/or formed using an additive manufacturing process, such as electron beam melting (EBM) or direct metal laser sintering (DMLS), to list only a few possibilities.

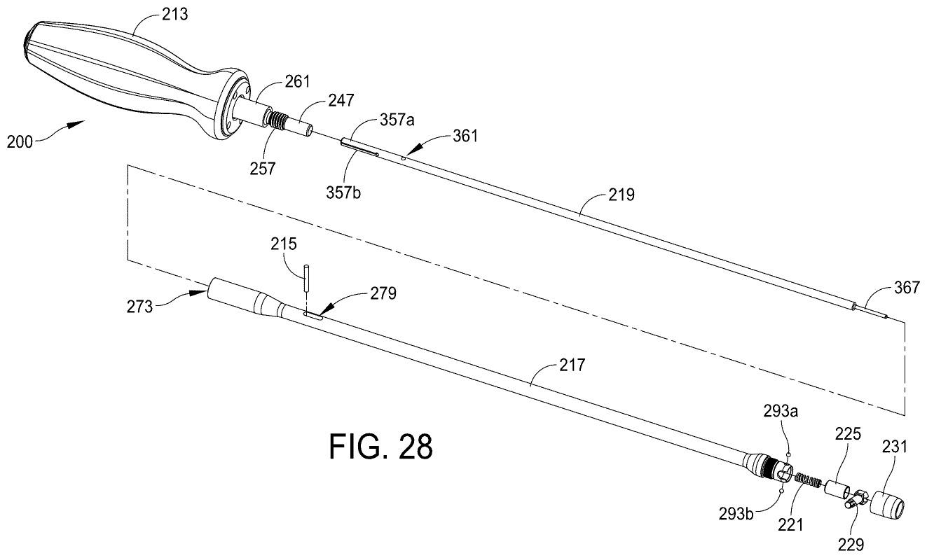

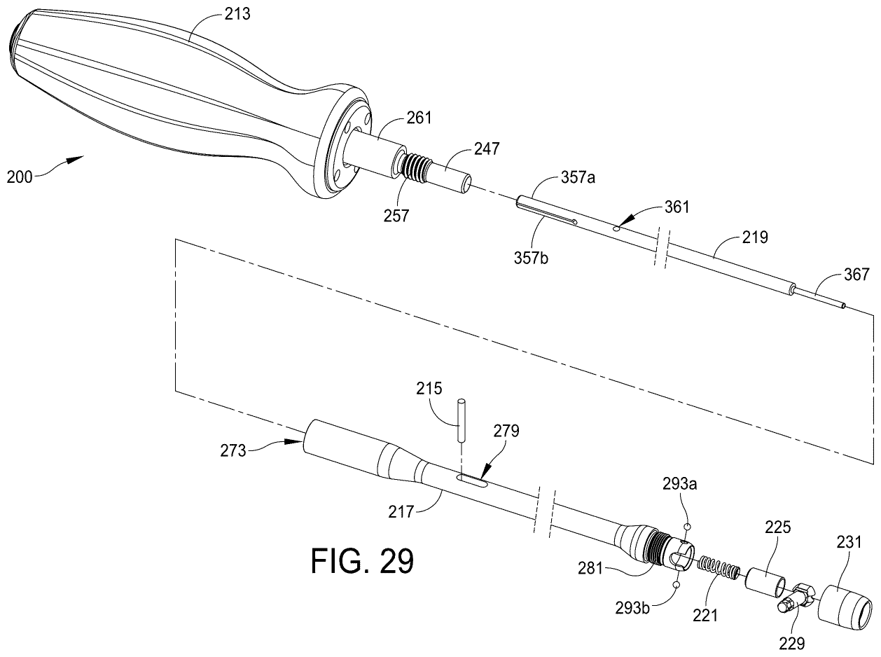

FIGS. 28-29 illustrate exploded views of surgical driver 200 in accordance with some embodiments. Surgical driver 200 may be sold already assembled, as illustrated in FIGS. 17-19, or come as a kit for a user to assemble as illustrated in FIGS. 28-29. It will be appreciated that surgical driver 200 may be disassembled and reassembled by a user for cleaning, maintenance, repair, or to exchange one or more parts. For example, a user may desire a different tip 229 with a different operating end 325 as discussed herein.

Surgical driver 200 is assembled by inserting rod 219 into aperture 273 of shaft 17. The securing member 247 of handle 213 is also into aperture 273 of shaft 217. Securing portion 257 of securing member 247 and securing feature 277 of shaft 217 engage and couple handle 213 to shaft 217. For example, securing portion 257 may be threaded and securing feature 277 is also threaded with respective threads such that securing portion 257 and securing feature 277 are removably coupled by the threads.

FIGS. 30-31 illustrate cross-sectional views of surgical driver 200 along detail B illustrated in FIG. 20 in accordance with some embodiments. Biasing member 221 is inserted into slot 297 of shaft 217. Anvil 225 is also inserted into slot 297 over biasing member 221 such that anvil 225 is biased against shaft 217 with biasing member 221 and fixation portion 367 of rod 219 is disposed within void 311. Tip 229 is inserted into slot 297 such that first end 313 of tip 229 engages surface 309 of anvil 225. Bearings 293a-b are then inserted into holes 289a-b of retaining feature 285 and into slots 321a-b of tip 229. Sleeve 231 is then slid over tip 229 and shaft 217 such that tip 229 and shaft 217 are disposed within void 341. With sleeve 231 installed, securing feature 345 of sleeve 231 engages second securing feature 281 of shaft 217 to couple sleeve 231 and shaft 217 together. For example, securing feature 345 and second securing feature 281 are threaded with respective threads such that securing feature 345 and second securing feature 281 are removably coupled by the threads.

Sleeve 231 constrains tip 229 within slot 297. Movement of tip 229 is facilitated by anvil 225 and bearings 293a-b such that a user can orient operating end 325 from a first angle to a second angle. Biasing member 221 also prevents tip 229 from moving without user input. For example, the force of biasing member 221 against anvil 225 keeps operating end 325 oriented at the angle set by the user. This feature allows the user to control the position and orientation of the tip 229. The intersection angle between the input shaft (i.e., shaft 217) and output shaft (i.e., tip 229) is also variable within a set upper and lower bounds. For example, the intersection angle may be set to ±30°, but it will be appreciated that this value may be increased or decreased as desired.

A user may fixate tip 229 along the longitudinal axis X of surgical driver 200 (as best illustrated in FIG. 18) by moving pin 215 into aperture 361 as discussed above. The user can free tip 229 to move to some angle A from the longitudinal axis X of surgical driver 200 (as best illustrated in FIG. 17) by moving pin 215 back towards handle 213 such that pin 215 is removed from aperture 361.

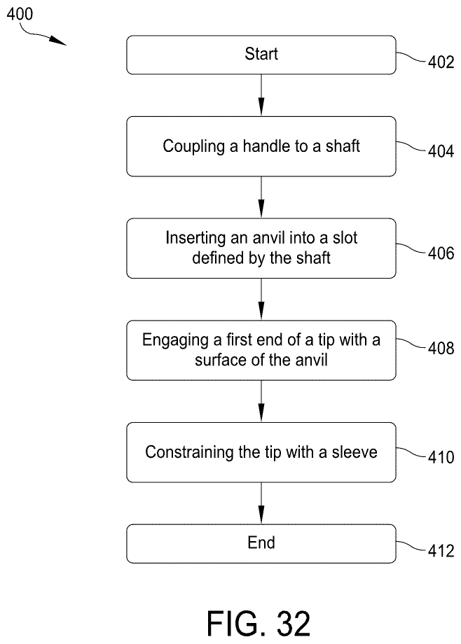

FIG. 32 illustrates an exemplary method 400 of assembling a surgical driver 10, 200 in accordance with some embodiments. Method 400 starts at block 402. At block 404, method 400 comprises coupling a handle 13, 213 to a shaft 17, 217. At block 406, method 400 comprises inserting an anvil 25, 225 into a slot 97, 297 defined by the shaft 17, 217. At block 408, method 400 comprises engaging a first end 113, 313 of a tip 29, 229 with a surface 109, 309 of the anvil 25, 225. At block 410, method 400 comprises constraining the tip 29, 229 with a sleeve 31, 231. The method 400 ends at block 412.

In some embodiments, method 400 comprises loading the tip 29, 229 with a biasing member 21, 221 disposed between the anvil 25, 225 and the shaft 17, 217. In some embodiments, method 400 comprises moving the tip 29, 229 from a first angle to a second angle facilitated by the anvil 25, 225. In some embodiments, method 400 comprises inserting a rod 219 into an aperture 273 of the shaft 217. In some embodiments, method 400 comprises locking the tip 229 with the rod 219. In some embodiments, method 400 comprises removing the rod 219 from the tip 229 such that the tip 229 is free to move.

### Features of the Disclosure

In some embodiments, a driver for transmitting off-axis torque may include a handle. The driver may include a shaft extending between a first end and a second end. The shaft may be coupled to the handle at the first end of the shaft. The second end of the shaft may define a void. The driver may include an anvil disposed within the void of the second end of the shaft. The driver may include a biasing member disposed within the void of the second end of the shaft between the shaft and the anvil. The biasing member may be configured to bias the anvil against the second end of the shaft. The driver may include a tip engaged with a surface of the anvil. The driver may include a sleeve coupled to the second end of the shaft such that the tip is constrained.

In some embodiments, the handle may extend between a first end and a second end, and may define a grip between the first end and the second end. In some embodiments, the grip may include at least one of a non-skid surface, a plurality of ridges, or a wrap. In some embodiments, the grip may be a different material than the first end and the second end of the handle. In some embodiments, the handle may include a securing member having a securing portion.

In some embodiments, the shaft may define a securing feature at the first end of the shaft. The securing feature of the shaft may be complementary to the securing portion on the handle such that the securing portion of the handle and the securing feature of the shaft are coupled together. In some embodiments, the securing portion of the handle and the securing feature of the shaft may each be threaded. In some embodiments, the second end of the shaft may define a second securing feature and the sleeve may define a complementary securing feature. In some embodiments, the second securing feature of the shaft and the complementary securing feature of the sleeve may be threaded. In some embodiments, the shaft may define a retaining feature at the second end of the shaft. In some embodiments, the retaining feature may define a plurality of holes each sized and configured to receive a respective bearing.

In some embodiments, the tip may define a plurality of slots at a first end that are each sized and configured to receive the respective bearing. In some embodiments, a second end of the tip may have one or more surface features. In some embodiments, the one or more surface features may be threads. In some embodiments, the one or more surface features may be formed in a shape of a bit. In some embodiments, the biasing member may be a spring. In some embodiments, the surface of the anvil may be curved. In some embodiments, the shaft may define an aperture therethrough. The aperture may be sized and configured to receive a rod. In some embodiments, the rod may extend between a first end and a second end, the first end may define an aperture configured to receive a pin. In some embodiments, the anvil may define an aperture therethrough and the tip may define a slot at a first end. The aperture of the anvil and the slot of the tip may be aligned and sized and configured to receive the second end of the rod to fixate the tip along a longitudinal axis of the shaft.

In some embodiments, a driver for transmitting off-axis torque may include a handle configured to transmit torque. The driver may include a shaft extending between a first end and a second end. The shaft may define an aperture between the first end and the second end. The shaft may be coupled to the handle at the first end of the shaft. The second end of the shaft may define a slot. The driver may include an anvil that may define an aperture disposed within the slot of the second end of the shaft. The driver may include a biasing member disposed within the slot of the second end of the shaft. The biasing member may be configured to bias the anvil against the second end of the shaft. The driver may include a tip defining a slot at a first end. The first end may be engaged with a surface of the anvil. The driver may include a rod disposed within the aperture of the shaft. The rod may be configured to fixate the tip at an angle along a longitudinal axis of the shaft. The driver may include a sleeve coupled to the second end of the shaft such that the tip is constrained.

In some embodiments, the rod may extend between a first end and a second end. The first end may define an aperture configured to receive a pin. In some embodiments, the handle may extend between a first end and a second end, and may define a grip between the first end and the second end. In some embodiments, the grip may include at least one of a non-skid surface, a plurality of ridges, or a wrap. In some embodiments, the grip may be a different material than the first end and the second end of the handle. In some embodiments, the handle may include a securing member having a securing feature.

In some embodiments, the shaft may define a securing feature at the first end of the shaft. The securing feature of the shaft may be complementary to the securing member on the handle such that the securing feature of the handle and the securing feature of the shaft are coupled together. In some embodiments, the securing feature of the handle and the securing feature of the shaft are each threaded. In some embodiments, the second end of the shaft may define a second securing feature and the sleeve may define a complementary securing feature. In some embodiments, the second securing feature of the shaft and the complementary securing feature of the sleeve may be threaded. In some embodiments, the shaft may define a retaining feature at the second end of the shaft. In some embodiments, the retaining feature may define a plurality of holes each sized and configured to receive a respective bearing.

In some embodiments, the tip may define a plurality of slots at a first end that are each sized and configured to receive the respective bearing. In some embodiments, a second end of the tip may have one or more surface features. In some embodiments, the one or more surface features may be threads. In some embodiments, the one or more surface features may be formed in a shape of a bit. In some embodiments, the biasing member may be a spring. In some embodiments, the surface of the anvil may be curved.

In some embodiments, a method may include coupling a handle to a shaft. The method may include inserting an anvil into a slot defined by the shaft. The method may include engaging a first end of a tip with a surface of the anvil. The method may include constraining the tip with a sleeve.

In some embodiments, the coupling of the handle to the shaft may be facilitated by a securing feature on the handle and a securing feature on the shaft. In some embodiments, the method may include loading the tip with a biasing member disposed between the anvil and the shaft. In some embodiments, the constraining of the tip may be facilitated by a plurality of bearings. In some embodiments, the method may include moving the tip from a first angle to a second angle facilitated by the anvil. In some embodiments, the method may include inserting a rod into an aperture of the shaft. In some embodiments, the method may include locking the tip with the rod. In some embodiments, the method may include removing the rod from the tip such that the tip is free to move.

Although the drivers, systems, kits, and methods have been described in terms of exemplary embodiments, they are not limited thereto. Rather, the appended claims should be construed broadly, to include other variants and embodiments of the drivers, systems, kits, and methods, which may be made by those skilled in the art without departing from the scope and range of equivalents.

1. A driver for transmitting off-axis torque comprising:
a handle;
a shaft extending between a first end and a second end, the shaft being coupled to the handle at the first end of the shaft, the second end of the shaft defining a void;
an anvil disposed within the void of the second end of the shaft;
a biasing member disposed within the void of the second end of the shaft between the shaft and the anvil, the biasing member configured to bias the anvil against the second end of the shaft;
a tip engaged with a surface of the anvil; and
a sleeve coupled to the second end of the shaft such that the tip is constrained.

2. The driver of claim 1, wherein the handle extends between a first end and a second end, and defines a grip between the first end and the second end.

3. The driver of claim 2, wherein the grip comprises at least one of a non-skid surface, a plurality of ridges, or a wrap.

4. The driver of claim 3, wherein the grip is a different material than the first end and the second end of the handle.

5. The driver of claim 1, wherein the handle comprises a securing member having a securing portion.

6. The driver of claim 5, wherein the shaft defines a securing feature at the first end of the shaft, the securing feature of the shaft being complementary to the securing portion on the handle such that the securing portion of the handle and the securing feature of the shaft are coupled together.

7. The driver of claim 6, wherein the securing portion of the handle and the securing feature of the shaft are each threaded.

8. The driver of claim 1, wherein the second end of the shaft defines a second securing feature and the sleeve defines a complementary securing feature.

9. The driver of claim 8, wherein the second securing feature of the shaft and the complementary securing feature of the sleeve are threaded.

10. The driver of claim 1, wherein the shaft defines a retaining feature at the second end of the shaft.

11. The driver of claim 10, wherein the retaining feature defines a plurality of holes each sized and configured to receive a respective bearing.

12. The driver of claim 11, wherein the tip defines a plurality of slots at a first end that are each sized and configured to receive the respective bearing.

13. The driver of claim 12, wherein a second end of the tip has one or more surface features.

14. The driver of claim 13, wherein the one or more surface features are threads.

15. The driver of claim 13, wherein the one or more surface features are formed in a shape of a bit.

16. The driver of claim 1, wherein the biasing member is a spring.

17. The driver of claim 1, wherein the surface of the anvil is curved.

18. The driver of claim 1, wherein the shaft defines an aperture therethrough, the aperture being sized and configured to receive a rod.

19. The driver of claim 18, wherein the rod extends between a first end and a second end, the first end defining an aperture configured to receive a pin.

20. The driver of claim 19, wherein the anvil defines an aperture therethrough and the tip defines a slot at a first end, the aperture of the anvil and the slot of the tip being aligned and sized and configured to receive the second end of the rod to fixate the tip along a longitudinal axis of the shaft.

21.-46. (canceled)
